(19) United States
(12) Patent Application Publication
(10) Pub. No.: US 2022/0155228 A1
(43) Pub. Date: May 19, 2022

(54) MULTI COMPARTMENT SENSING UNIT COMPRISING BIOSENSORS

(71) Applicant: YISSUM RESEARCH DEVELOPMENT COMPANY OF THE HEBREW UNIVERSITY OF JERUSALEM LTD., Jerusalem (IL)

(72) Inventors: Aharon AGRANAT, Mevasseret Zion (IL); Shimshon BELKIN, Kiryat Ono (IL); Yossef KABESSA, Nof-Ayalon (IL); Etai SHPIGEL, Mazkeret Batya (IL); Benjamin SHEMER, Jerusalem (IL); Tal ELAD, Jerusalem (IL)

(21) Appl. No.: 17/528,410

(22) Filed: Nov. 17, 2021

Related U.S. Application Data
(60) Provisional application No. 63/114,816, filed on Nov. 17, 2020.

(51) Int. Cl. G01N 21/64; C12Q 1/02

(52) U.S. Cl. CPC G01N 21/6428; G01N 27/02; G01N 2021/6441; C12Q 1/02

(57) ABSTRACT

A sensing unit is disclosed. The sensing unit includes: a first compartment which includes a first group of polymer beads carrying genetically engineered cells configured to produce a reporter when exposed to a target material; and a first detector configured to detect the reporter; and a second compartment having: a second group of the polymer beads carrying the genetically engineered cells configured to produce the reporter when exposed to the target material; and a second detector configured to detect the reporter. The second compartment is impenetrable to the target material and the first compartment is penetrable to the target material.

### CROSS-REFERENCE TO RELATED APPLICATIONS

This application claims the benefit of priority of U.S. Provisional Application Ser. No. 63/114,816, filed Nov. 17, 2020, which is hereby incorporated by reference in its entirety.

### FIELD OF THE INVENTION

The present invention relates generally to biosensors.

### BACKGROUND OF THE INVENTION

Biosensors (e.g., whole-cell biosensors) harness the ability of live cells to continuously monitor their microenvironment and to respond to local environmental changes by expressing specific gene sets. Such cells can be genetically “tailored” to respond to diverse types of chemical, physical, and biological stimuli.

In recent years, a considerable effort has been devoted to genetic engineering of cells to enable the detection of trace quantities of specific chemicals, or groups of chemicals sharing common chemical structure and/or properties or sharing a biological activity. The response of living cells to a target material can be dose-dependent and may be manifested by activating at least one of several reporting mechanisms.

Accordingly, living cell biosensors may provide an accurate, robust, simple and in situ method for detecting and quantifying target materials. These cell-based biosensors can be engineered to detect many different compounds and environmental conditions.

### SUMMARY OF THE INVENTION

Aspects of the invention are directed to a sensing unit comprising: a housing said housing comprises:
a first compartment comprising:
   a first group of polymer beads comprising genetically engineered living cells configured to produce a reporter when exposed to a target material; and
   a first detector configured to detect said reporter; and
a second compartment comprising:
   a second group of said polymer beads comprising said genetically engineered living cells configured to produce said reporter when exposed to said target material; and
   a second detector configured to detect said reporter.

In some embodiments, the second compartment is impenetrable to said target material and said first compartment is penetrable to said target material. In some embodiments, the first group of polymer beads and said second group of polymer beads comprise the same number of beads, and wherein all beads in both groups comprise an equal or similar distribution of said genetically engineered living cells from the same production batch.

In some embodiments, the second compartment comprises a cover, blocking said second group of said polymer beads from being exposed to said target material. In some embodiments, the first compartment comprises an opening configured to allow said first group of polymer beads but not said second group of polymer beads to be exposed to said target material. In some embodiments, the housing further comprises a blocking element for blocking at least said first compartment from external biotic and abiotic cues excluding said target material, said second compartment from external biotic and abiotic cues, or both.

In some embodiments, the sensing unit further comprises, a control circuit configured to receive a signal from said first detector and said second detector; and a communication unit configured to communicate with an external computing device.

In some embodiments, the reporter is luminescent and said first and said second detectors are luminescence detectors configured to detect an emitted bioluminescent light. In some embodiments, the reporter changes electrical resistivity of a polymer bead and said first and said second detectors are any device measuring any electrical property including but not limited to: amperemeters, ohmmeters, LCR meters, and voltmeters. In some embodiments, the reporter is bio-chemiluminescence or bioluminescence.

In some embodiments, the sensing unit is designed to be placed on a ground surface.

Additional aspects of the invention are directed to a sensing unit comprising:
a housing wherein the housing comprises;
   a first compartment comprising:
      an inspection container comprising genetically engineered living cells configured to produce a reporter when exposed to a target material; and
      a first detector configured to detect said reporter; and
   a second compartment comprising:
      a control container comprising said genetically engineered living cells configured to produce said reporter when exposed to said target material; and
      a second detector configured to detect said reporter.

In some embodiments, the control container comprising a known concentration or amount of said target material. In some embodiments, the inspection container and the control container comprise the same amount or concentration of genetically engineered living cells from the same production batch.

In some embodiments, the housing further comprises a blocking element for blocking said first compartment from external biotic and abiotic cues, said second compartment from external biotic and abiotic cues, or both.

In some embodiments, the sensing unit further comprises: a control circuit configured to receive signals from said first detector and said second detector; and a communication unit configured to communicate with an external computing device.

In some embodiments, the reporter luminescent and said first and said second detectors are luminescence detectors configured to detect an emitted bioluminescent light. In some embodiments, the reporter changes electrical resistivity of a material held in said inspection container and said control container and said first and said second detectors are devices capable of measuring any electrical property selected from amperemeters, ohmmeters, LCR meters, and voltmeters. In some embodiments, the reporter chemiluminescence or bioluminescence.

Additional aspects of the invention are directed to a method of sensing an environ target material using genetically engineered living cells, comprising:
receiving a first signal indicative of a reporter, wherein said first signal is from a first detector, said first detector is included in a first compartment comprising, a first group of polymer beads comprising genetically engineered living cells configured to produce a reporter when exposed to said target material;
receiving a second signal indicative of a spontaneous reaction or noise, wherein said second signal is from a second detector, said second detector is included in a second compartment, comprising, a second group of polymer beads comprising said genetically engineered living cell; and
filtering said first signal using said second signal.

In some embodiments, the second compartment is impenetrable to said target material and said first compartment is penetrable to said target material. In some embodiments, first group of polymer beads and said second group of polymer beads comprise the same number of beads, and wherein all beads in both groups comprise an equal or similar distribution of said genetically engineered living cells from the same production batch.

In some embodiments, first detector and the second detector are configured to detect the reporter.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is an illustration of a genetically engineered bacterium, modified with a reporter gene that emits bioluminescent photons upon exposure to target material (activation, production);

FIG. 1B is an illustration of several prior art reporting mechanisms of a genetically engineered living cell;

FIG. 2A is an illustration of a sensing unit comprising genetically engineered living cells, according to some embodiments of the invention;

FIG. 2B is a detailed illustration of a non-limiting example of a sensing unit comprising genetically-engineered bioluminescent bacteria, according to some embodiments of the invention;

FIG. 2C is an illustration of cassettes comprising beads carrying bacteria according to some embodiments of the invention;

FIG. 2D is an illustration of an electric circuit of the sensing unit of FIG. 2B, according to some embodiments of the invention;

FIG. 3A is an illustration of another sensing unit comprising genetically engineered living cells, according to some embodiments of the invention;

FIG. 3B is a detailed illustration of another nonlimiting example of a sensing unit comprising genetically-engineered bioluminescent bacteria, according to some embodiments of the invention;

FIG. 3C is an illustration of a process for calculating standard ratio (SR) using a sensing unit of FIG. 3B;

FIG. 4A is a block diagram of a system of sensing units according to some embodiments of the invention;

FIG. 4B is an illustration of the placement of a system of sensing unit in the field, according to some embodiments of the invention;

FIG. 5 is a block diagram, depicting a computing device according to some embodiments;

FIG. 6 is a flowchart of a method of sensing an environ target material using genetically engineered living cells according to some embodiments of the invention;

FIG. 7 is a flowchart of a method of quantitively detecting an amount or concentration of a target material in a container, according to some embodiments of the invention;

FIG. 8A shows graphs demonstrating the response of identical inspected solutions having the same amount of target material and the same amount of bacteria according to some embodiments of the invention;

FIG. 8B are SR values and the output signal values as a function of batch (graph (a)) and temperature (graph (b)) according to some embodiments of the invention; and

FIG. 8C is a calibration curve for SR according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are outlined in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of the same or similar features or elements may not be repeated.

In some embodiments, provided herein is a sensor unit built of compartments wherein at least two compartments comprise beads carrying genetically modified bacteria. The content of beads and bacteria in the two compartments is similar and/or identical.

In some embodiments, bacteria are used as the core sensing elements of an optoelectronic (OE) circuit. In some embodiments, the bacteria serve as the input devices of an OE circuit in which they operate in unison with the OE circuit, producing a signal which reports the presence and/or quantity of a target compound or material. In some embodiments, the phrases “target compound” and “target material” are synonymous.

In some embodiments, the “core sensing element” comprises the bacteria which operate as the analog interface between the physical environment and the digital environment to which the sensor unit serves as the gate.

In some embodiments, the present invention provides a chemical sensing device which merges hardware, software, and “wetware”. As such, the device provides a unique combination of performance envelope (sensitivity, variability in sensing different target materials, etc.) with modes of deployment.

Biosensors

In some embodiments, the genetically modified bacteria or sensing bacteria may be considered to act as “minuscule biochemical laboratories” that are genetically engineered to respond to a specific target compound/material (or a specific group of target materials, or any specific environmental parameter/s) by producing an output signal.

The target material in the sample to which they are exposed, a promoter to be directly or indirectly activated thereby driving the expression of the reporter which leads to the activation of the reporting mechanism. In some embodiments, the reporter is a fluorescent protein or emits bioluminescence, produces coloration, and/or impacts the electric charge or conductivity. The detection of the reporting signal that the reporting mechanism produced is done by the circuit in which the bacteria are embedded. This is done, in some embodiments, in the domain of optoelectronics).

In one embodiment, a biosensor is composed of genetically modified bacteria. In one embodiment, a biosensor is composed of a carrier and genetically modified bacteria. In one embodiment, a biosensor is composed of a carrier such as beads to which genetically modified bacteria are adhered thereto or encapsulated in.

In one embodiment, “bacteria” comprises genetically modified bacteria. In one embodiment, genetically modified bacteria have the capacity to be induced to produce a reporter. In one embodiment, a reporter is a protein expressed in the genetically modified bacteria as a consequence of exposure to a predefined set of conditions. In one embodiment, a reporter gene is conditionally expressed due to its coupling to a cellular regulatory circuit. In some embodiments, the cellular regulatory circuit is a DNA sequence introduced to bacteria or modified in the bacteria thereby rendering the bacteria genetically modified. In some embodiment, the regulatory circuit or a segment of the regulatory circuit is a bacterial DNA sequence inherently present in the bacteria. In some embodiment, the regulatory circuit or a segment of the regulatory circuit is a bacterial DNA sequence introduced to the bacteria to drive the expression of the reporter.

The regulatory circuit such as but not limited to a central regulatory circuit may comprise a regulatory protein which senses the presence of the target compound and activates gene expression from a dedicated activator site (or switch) in the bacterial chromosome or in an exogenous DNA molecule within the bacteria that is next to a promoter.

In some embodiments, duplicating the DNA segment comprising this switch and promoter and fusing it to a promoterless reporter gene, expression of the reporter is brought under the control of the regulatory protein. Reporter synthesis will thus start when the bacteria encounter or sense or are in contact with the target compound (effector compound) that the regulatory protein recognizes.

In some embodiments, the reporter circuit is uncoupled and comprises a compound-activated regulatory circuit but in heterologous bacteria that cannot metabolize the detected target compound. In some embodiments, the reporter circuit is uncoupled from any constraints or additional control mechanisms that the bacteria host may exert on the switch and promoter.

In some embodiments, the bacteria/biosensor is designed to operate as toxicity- or genotoxicity- or stress-responsive bacteria. In some embodiments, the bacteria comprise a selected duplicated gene promoter from a stress response network, known to be activated by a desired condition or target compound, is fused to the reporter gene, and becomes embedded in the existing network of the bacteria.

In some embodiments, bacteria of the invention are characterized by coupling the sensing of a target compound to reporter protein production. In some embodiments, bacteria of the invention are characterized in that an existing signaling pathway is monitored not by its native response but by an artificial output.

In some embodiments, the expression of a gene (or group of genes) encoding the desired output, termed the ‘reporter protein’, is artificially brought under the control of a sensory-regulatory system or network with the required specificity. In some embodiments, the amount, or activity of the reporter protein produced is taken as a proxy for the bacterial response to the target compound. In some embodiments, the reporter protein is not present in the native bacteria. In one embodiment, reporter proteins to be used in the bacteria are readily available to a person of skill in the art.

In some embodiments, bacteria, as described herein, are genetically modified with a target compound-specific circuit and/or reporter. In some embodiments, regulatory control is achieved by a single sensor-regulator protein that is responsible for the intracellular perception of the targeted compound and subsequent gene expression from one or a few key promoters. In some embodiments, the reporter circuit is assembled from the DNA segments for the key promoter and the activation site of the sensor-regulator protein fused to a reporter gene, with or without an extra copy of the gene encoding the regulatory protein itself.

In some embodiments, target compound detection by bacteria is based on the direct or indirect intracellular reaction of catabolic regulatory proteins with the metabolized target compound. In some embodiments, different target compound recognition specificities of the catabolic regulatory proteins are exploited for reporter design, such as but not limited to construction of a regulatory protein from the LysR-type transcriptional activator family, such as NahR, which controls reporter expression. In some embodiments, the Xy1R-DmpR subclass of NtrC transcriptional activators is used. In some embodiments, a regulatory protein is synthetically engineered in order to provide sufficient sensitivity and specificity to the target compound.

In some embodiments, the biosensor and/or bacteria is designed according to the teachings of van der Meer JR. and Belkin S. 2010 (van der Meer J R, Belkin S. Where microbiology meets microengineering: design and applications of reporter bacteria. Nat. Rev. Microbiol. 2010 July; 8(7):511-22. doi: 10.1038/nrmicro2392. PMID: 20514043.) which is hereby incorporated by reference in its entirety. In some embodiments, the biosensor and/or bacteria is designed according to the teachings of Belkin S. 2003 (Belkin S. Microbial whole-cell sensing systems of environmental pollutants. Curr. Opin. Microbiol. 2003 June; 6(3):206-12. doi: 10.1016/s1369-5274(03)00059-6. PMID: 12831895.) which is hereby incorporated by reference in its entirety.

As used herein genetically engineered living cells may include any type of living cells that could be genetically modified, for example, bacteria, eukaryotic cells (including but not restricted to yeast cells, plant cells, mammalian cells), or any solitary living cell.

As used herein, a reporter gene is a gene whose expression (used herein as a “reporter”) is readily detectable by a sensor as described herein. The reporter gene's expression (i.e., the reporter) is regulated or controlled by a regulatory region or element designed to be activated by a specific compound or material such as the target material. The reporter gene is a nucleic acid sequence encoding for the reporter. In one embodiment, a reporter is an easily assayed protein. The reporter gene “reports” on the presence of the target material and may provide a quantitative “report”. The introduced reporter gene is driven by a promoter, or a “transgene” activated directly or indirectly by the target material. The reporter gene is cloned downstream of a regulatory region (e.g., promoter/enhancer) that is usually responsible for the controlled expression of a specific gene.

Thus, by introducing a reporter gene driven by a promoter activated directly or indirectly by the target material one can monitor the presence and/or the amount of the target material. In some embodiments, reporter genes include but are not limited to lacZ, phoA, β-glucuronidase gene, alkanal monooxygenase gene, bacterial luciferase gene cassette (luxCDABEG) or firefly luciferase gene (luc)

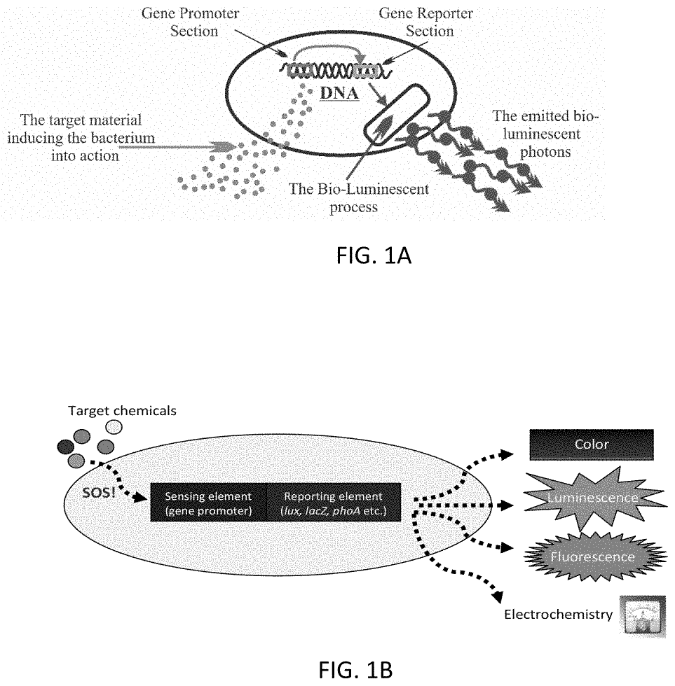

In a non-limiting example illustrated in FIG. 1A, an engineered biosensing bacterium harbors a genetic element such as a plasmid comprising a sensing/activating element that detects the presence of the target material and possibly activates a reporter. The reporter may include a physical/optical characteristic which enables its detection.

Possible reporting mechanisms may include, (i) the generation of an electrical charge; (ii) the emission of light produced by bioluminescence; (iii) chromogenic (color change) response; (iv) the production of fluorescent molecules (e.g., Green Fluorescence Protein (GFP)) and the like. Some non-limiting examples of such mechanisms are illustrated in FIG. 1B.

### Sensing Unit for Detecting a Target Material

Embodiments of the present invention disclose a multi-compartment sensing unit wherein at least 2 compartments comprise biosensors and a method and a system for using such sensing units for detecting target material(s)/compound(s) and/or other specific physical or chemical conditions in the surrounding of the sensing unit. A sensing unit according to embodiments of the invention can be used as a standalone autonomous unit. For example, a sensing unit can be placed on a ground surface for sensing target material/conditions in the ground, can float on an aqueous solution or a body of water (e.g., a sewage water reservoir) for detecting a target material, exposed to ambient air, and the like. The sensing unit may be loaded with living cells, such as but not limited to, bacteria that are genetically engineered, wherein the cells generate detectable/quantifiable products/reporter in the presence of the target material.

The target material may be any material that can induce directly or indirectly the expression of the reporter (or activate its promoter). In some embodiments, the target material is present in the form of a mixture or a solution (e.g., mixed with soil), dissolved in a liquid (e.g., dissolved in an aqueous solution), and/or included in a gas phase (e.g., included in the air). In a non-limiting example, the target material is a contaminant, for example, an oil contaminant, a chemical contaminant, a biological contaminant, and the like. In another non-limiting example, the target material is a contaminant, or an undesired material dissolved in an aqueous solution, for example, a poison or a toxic material present in water. In yet another non-limiting example, the target material may be undesired gas.

In some embodiments, the sensing unit is configured to detect minute amount/concentration (e.g., traces of less than 1 wt. % or 0.1 wt. %) of the target material. Some non-limiting specific examples of target materials may include: antibiotics, drug, poison, pollutants, pesticides (e.g., insecticides, herbicides, fungicides, etc.). Other non-limiting examples may include, chemicals released by insects, factories and other pests as a means for early detection.

In some embodiments, the sensing unit can detect a compound or a group of compounds sharing a common structure (e.g., halogenated aromatics, heavy metals, etc.), or a common chemical characteristic (e.g. oxidative) for example, for monitoring/mapping of environmental pollution. In some embodiments, the sensing unit can detect a group of compounds sharing biological activity (e.g., oxidants, DNA damaging agents, membrane damaging agents, endocrine disrupting compounds, etc.), for example, for monitoring/mapping of environmental pollution.

In some embodiments, the sensing unit may detect, for example, heavy metals toxicity, for early warning against the accidental presence of heavy metals in, soil, water, air, or food. In some embodiments, the sensing unit may detect the accidental presence of mutagenic/carcinogenic compounds in water, air, or food.

In some embodiments, the sensing unit provides the required conditions for supporting the life of the genetically engineered living cells. In some embodiments, the sensing unit includes polymeric beads or capsules comprising the living cells such as bacteria.

In a non-limiting example, a bacteria may be encapsulated in or attached to a bead comprising a polymer such as alginate. For this purpose, a bacterial culture is mixed with liquid alginic acid (i.e., alginate) along with any other dissolved medium components. The mixture is added dropwise to a solution of calcium chloride. Upon the interaction with the calcium ions, the drop immediately solidifies into a transparent bead having substantially the drop size. Similar drop sizes, from similar production batches, may provide substantially similar beads having substantially the same concentration of living cells, the same type of cells, and at the same developmental stage.

Therefore, each bead or all beads within the sensing unit is/are expected to generate a similar reporting output upon expose to the target material/compound. In another embodiment, each bead or all beads within the sensing unit is/are expected to generate a similar background reporting output when not exposed to the target material/compound. In some embodiments, the first group of beads is used for detection of the target material while the second group of beads is used for detection of background spontaneous generation of reporter and/or noise and filtering these effects from the detected reporter generated upon the exposure to the target material. In some embodiments, the first and second groups of beads are equivalent or identical with respect to the number of beads, the identity and culture history of the bacteria, the bacterial concentration in or on each bead and the bacterial batch.

Therefore, a sensing unit may provide accurate detection of the presence of the target material, after filtering out detection of spontaneous/background generation of a reporter. For this purpose, the sensing unit may include at least two compartments loaded with equal or similar number of beads, from the same production batch, a first group of beads for detecting and/or quantifying the target material/compound and a second group of beads for detecting the background spontaneous generation of a reporter. In some embodiments, the detected background/spontaneous generation is filtered out from the detection of reporter generated as a consequence to exposure to the target material. In some embodiments, the same amount of background spontaneous generation of a reporter is expected to occur in all beads from both compartments under the same conditions, for example, following equal temperature change. Accordingly, if one compartment is penetrable to the target material and the other compartment is impenetrable to the target material, the living cells in the other compartment may serve as reference cells for the spontaneous/non-specific generation of the reporter.

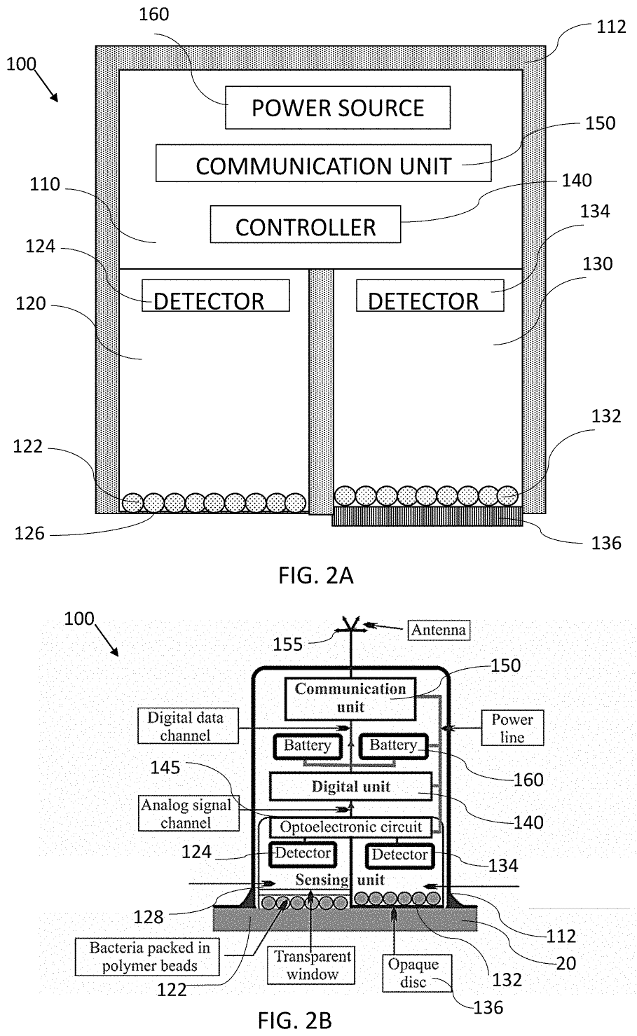

Reference is now made to FIG. 2A which is an illustration of a sensing unit comprising genetically engineered living cells, according to some embodiments of the invention. In some embodiments, sensing unit 100 includes a housing 110 comprising a first compartment 120 and a second compartment 130. In some embodiments, first compartment 120 includes a first group of polymer beads 122 comprising genetically engineered living cells configured to produce a reporter when exposed to a target material, and a first detector 124 configured to detect the reporter. In some embodiments, second compartment 130 includes a second group of polymer beads 132 comprising the same genetically engineered living cells, and a second detector 134 configured to detect the reporter.

In some embodiments, all beads are substantially the same as described herein and may comprise nutrients and water for supporting the viability of the cells. In some embodiments, the cells may be immobilized in a polymeric hydrogel that may serve as the physical support/entrapment of the cells. Possible suitable polymeric hydrogels may include (but are not limited to) agar-agar, agarose, alginate, and polyvinyl alcohol. The polymeric matrix may also include dissolved molecules that will serve as nutrients for the living cells, as well as additional components that may help with water retention and/or enhance the activity of the cells. One such potential additive is polyacrylic acid.

In some embodiments, all beads (or any other capsules comprising the living cells) in both first compartment 120 and second compartment 130 include an equal or similar distribution of the genetically engineered cells/bacteria from the same production batch. Furthermore, each compartment may include a similar number of such beads/capsules. Accordingly, both compartments possess the same biosensing ability/capacity under the same conditions and the same spontaneous behavior.

In one embodiment, cells are bacteria. In one embodiment, engineered or genetically engineered cells are engineered or genetically engineered bacteria.

In a first non-limiting example, the reporter may be luminescent and the first detector 124 and second detector 134 are luminescence detectors configured to detect an emitted bioluminescent light, as discussed in the nonlimiting example, of FIG. 2 B. In a second non-limiting example, the reporter may have chemiluminescence or bioluminescence. In a third non-limiting example, the reporter changes the electrical resistivity of a polymer bead or produced electricity, and first detector 124 and second detector 134 are selected from any device or sensor measuring an electrical property including but not limited to: amperemeters, ohmmeters, LCR meters, voltmeters, or any device configured to measure, conductivity, resistivity, impedance, capacitance and the like. In a fourth non-limiting example, the reporter is a chromogenic (color change) reporter, and first detector 124 and second detector 134 are selected from a camera and an optical detector.

In some embodiments, second compartment 130 is impenetrable to the target material and first compartment 120 is penetrable to the target material. In some embodiments, second compartment 130 comprises a cover 136, blocking the second group of polymer beads 132 from being exposed to the target material. For example, cover 136 may include or may be made from opaque material or a filter blocking any light from entering second compartment 130. In yet another example, cover 136 may seal compartment 130 from the environment or from external moisture, isolate compartment 130 from the surrounding temperature, and the like. Accordingly, the second group of polymer beads 132 may be substantially isolated for any biotic and/or abiotic cues as opposed to the first group of polymer beads 122 which are exposed to the environment.

In some embodiments, first compartment 120 includes an opening 126 configured to allow the first group of polymer beads 122 but not the second group of polymer beads 132 to be exposed to the environment and thereby potentially, to the target material. In some embodiments, opening 126 is located in compartment 120, thereby enabling compartment 120 to contact the environment/target material. For example, opening 126 may allow the first group of polymer beads 122 to come in contact with soil/water/air comprising the target material when device 100 is placed on the land/water or exposed to the air.

In some embodiments, housing 110 further includes a blocking element 112 for blocking at least first compartment 120 from external biotic and/or abiotic cues excluding the target material, blocking second compartment 130 from external biotic and/or abiotic cues, or both. In some embodiments, blocking element 112 may block only the first compartment 120 from all sides excluding opening 126. In some embodiments, blocking element 112 includes cover 136 thus blocking second compartment 130 from any external biotic and/or abiotic cues. In some embodiments, blocking element 112 may cover a portion of housing 110, for example, the portion comprising the beads and the detectors. A non-limiting example of such a blocking element is given in FIG. 2B. Blocking element 112 and/or cover 136 may allow using sensing unit 100 one the field, while still maintaining accurate readings from detectors 124 and 134, due to the blocking of the external biotic and abiotic cues.

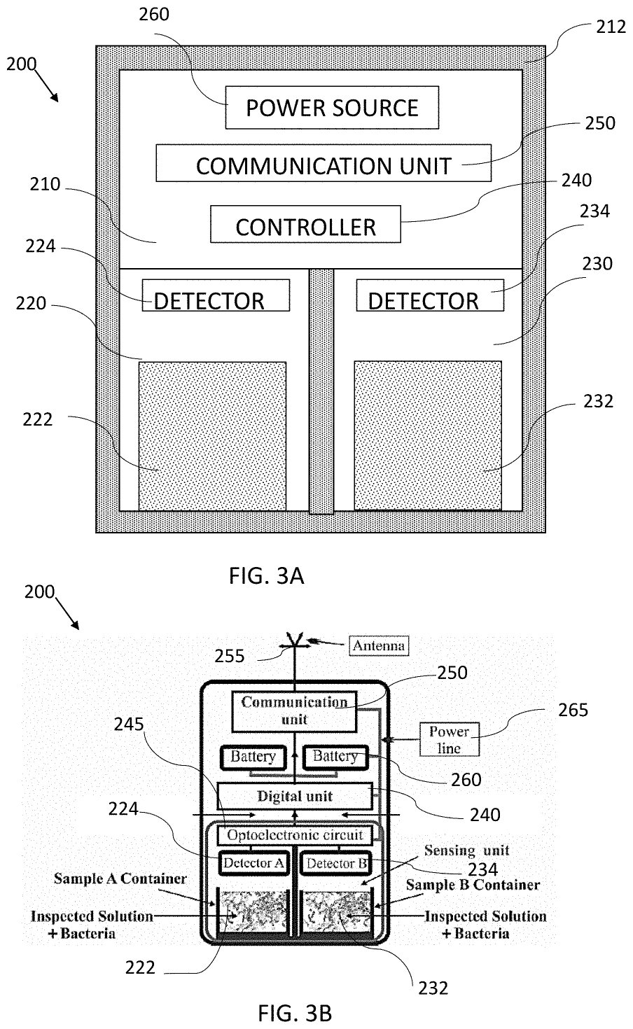

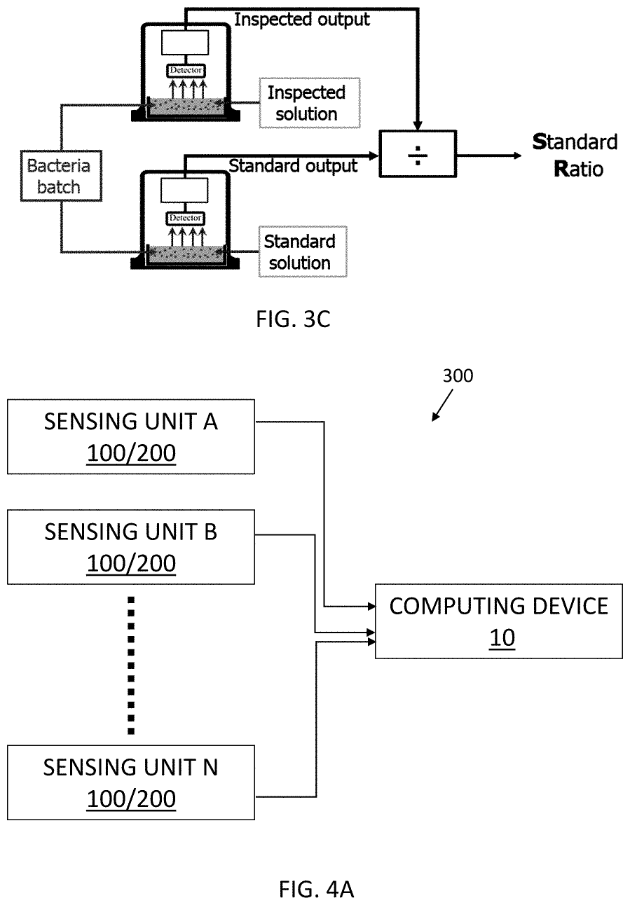

In some embodiments, sensing unit 100 includes a control circuit 140 configured to receive and process signals from first detector 124 and second detector 134. In some embodiments, control circuit 140 includes additional components, such as signal amplifiers (e.g., low noise amplifiers, difference amplifiers, and the like), a processing unit (e.g., a chip), and the like. In some embodiments, sensing unit 100 includes a communication unit 150 configured to communicate with an external computing device (e.g., computing device 10 illustrated and discussed in FIGS. 4A and 5) and a power source 160 (e.g., a battery). In some embodiments, communication unit 150 may include any wireless or wired module.

In some embodiments, unit 100 may further include one or more heating and/or cooling elements. As known in the art, the activity of the sensing living cells is temperature-dependent. Namely, the living cells are operable within a restricted temperature range, and temperature stability is a prerequisite for reproducible detection characteristics. Therefore, one or more heating/cooling elements (not illustrated) may be integrated into housing 110.

Reference is now made to FIG. 2B which is an illustration of a non-limiting example of sensing unit 100 according to some embodiments of the invention. Sensing unit 100 of FIG. 2B includes two compartments 120 and 130 each comprising a corresponding group 122 and 132 of polymeric beads loaded with genetically engineered bacteria, configured to generate a reporter when exposed to a target material. In the non-limiting example illustrated in FIG. 2B, the reporter is luminescent and first detector 124 and second detector 134 are luminescence detectors configured to detect an emitted bioluminescent light. In some embodiments, each compartment includes a similar number of beads, and all beads in both groups comprise an equal or similar distribution of the genetically engineered bacteria from the same production batch. In some embodiments, the beads include all the necessary life maintaining conditions for supporting the bacteria, as discussed hereinabove.

In some embodiments, the beads may be packed in bead cassette/cartridges. In some embodiments, each sensing unit contains two cassettes, a first cassette 121 comprising first group 122 of beads and a second cassette 131 comprising second group 132 of beads, which serves the reference beads. Both cassettes may be loaded with beads that contain bacteria genetically engineered to sense a specific target material. An illustration of cassettes 121 and 131 is given in FIG. 2C. In some embodiments, each cassette in a pair of cassettes may be loaded with the same bacteria (or any other living cells) genetically engineered to sense a specific target material. Accordingly, a selection of different pairs of cassettes, each pair for sensing different target material, is provided with each detecting unit 100, allowing using the same sensing unit for detecting different target materials. In some embodiments, the cassettes are positioned at the bottom of sensing unit 100 and are designed to be easily mounted to enable fast and convenient adaptation of sensing unit 100 to sense different target materials.

In some embodiments, first cassette 121 may include opening 126 allowing the first group of beads 122 to be exposed to the target material. In some embodiments, first cassette 121 may further include a transparent window 128, allowing bioluminescent photons from the reporter of the first group of beads 122 to reach first detector 124. In some embodiments, second cassette 134 may include an opaque cover (e.g., disc) 136 configured to block the second group of beads 132 from any external biotic and abiotic cues.

In the non-limiting example illustrated in FIG. 2C, cassettes 121 and 131 are built as two complementing half circles. Cassettes 121 and 131 may be mounted adjacent to each other at the bottom of the sensing unit 100. In some embodiments, the beads in cassette 121 are glued to transparent window 128. In some embodiments, the glue is transparent, hydrophilic, and non-toxic to the bacteria. In some embodiments, the beads in cassette 131 are glued on top of opaque plate 136 so that when the sensing unit is placed on the ground (or floating on liquid), the beads are completely separated from the environment such as ground environment or liquid environment. A partition between the cassettes prevents light generated in both cassettes to reach the detectors in the other compartment. It also prevents vapors released from the ground beneath sensing unit 100 to reach the beads in second cassette 131 and activate them.

In some embodiments, heating and/or cooling units may be integrated into cassettes 121 and 131 for controlling the temperature of the bacteria in the beads.

In some embodiments, in other to minimize or block external biotic and abiotic cues coming from the ground/surrounding of sensing unit 100, a skirt-like blocking element 112 may be added to housing 110. Therefore, when sensing unit 100 is placed on the ground or floating on water, opening 126 is exposed only to the ground/water and not to any other external biotic and abiotic cues.

For sensing the bioluminescent light, sensing unit 100 of FIG. 2B may include two luminescence detectors 124 and 134 configured to detect the emitted bioluminescent light. In a non-limiting example, the wavelength at which the bioluminescence emission is maximal is centered around 490 nm. Therefore, luminescence detectors 124 and 134 may be configured to detect photons having 490 nm wavelength. In some embodiments, the emitted light provides a weak signal (i.e. in the pico-watts range) therefore, requires amplification.

Sensing unit 100 may further include an electric circuit 140, which may include an optoelectronic (OE) circuit 145, or any other suitable circuit, that detects and amplifies the optical signals emitted by the bacteria. OE circuit 145 may produce an analog electronic signal that is proportional to the difference between the optical signals emitted by the bacteria (or the living cells) in first compartment 120 and second compartment 130. OE circuit 145 may be designed to exhibit high responsivity at low input light levels (i.e. in the pico-watts range). OE circuit 145 is designed to generate minimal internal noise, by minimizing the effect on the measurement accuracy due to variations in the signals produced by different batches of bacteria, and different environmental operation conditions such as temperature, humidity, and ventilation. In addition, the design of OE 145 circuit may take into account variations in the performance of different electronic components due to variations in the fabrication process, and changes in the performance of the components that occur over time.

A non-limiting example for OE circuit 145 is illustrated in FIG. 2D. OE circuit 145 is designed to operate, in parallel, two measurement channels: (i) a first channel in which the bacteria, included in compartment 120, are exposed to a substance 20 (e.g. ground surface, water) which may contain the target material; and (ii) a second channel that contains bacteria, included in second compartment 130, for detecting light emitted due to spontaneous bioluminescence or noise. The second channel operates subject to environmental conditions that are identical to those at which the first channel operates.

In some embodiments, OE circuit 145 may be constructed according to several different architectures. The non-limiting example illustrated in FIG. 2D adopts the concept of “differential architecture”, sometimes referred to as “balanced architecture”. OE circuit 145 may include two low noise amplifiers 144 each configured to amplify the signals received from each one of detectors 124 and 134 and a differential amplifier 146 configured to produce an analog electric signal that is proportional to the difference between the two signals. In some embodiments, OE circuit 145 detects simultaneously but separately the two optical signals generated by the bacteria in each compartment, amplifies them, reduces the second from the first, and produces the analog electric signal.

Referring back to FIG. 2B, which shows additional components of sensing unit 100. Sensing unit 100 may further include a digital unit 140 configured to process the output signal received from OE circuit 145 and optionally send the signal to an external computing device via a communication unit 150. Communication unit 150 may include a wireless communication module and an antenna 155 for wirelessly communicating with the external computing device. In some embodiments, sensing unit 100 may further include one or more batteries 160 for providing electrical power to the various components of sensing unit 100.

In some embodiments, the structure of sensing unit 100 (as illustrated and discussed in FIGS. 2A-2D) allows each sensing unit to act as an independent, autonomous, standalone unit.

### Sensing Unit for Quantitative Detection of a Target Material

Some additional aspects of the invention may be directed to a sensing unit that may conduct a quantitative detection of the target material. The quantitative detection may be conducted by a multicompartment sensing unit that includes biosensing living cells. The quantitative detection may be achieved by loading one compartment (e.g., the control compartment) with a known amount, or a known concentration of the target material, exposing the other compartment to the target material, and comparing the expressions of generated reporter between the two compartments.

In some embodiments, to quantitatively detect an amount of target material using biosensors, several drawbacks of these sensors may be overcome. As discussed hereinabove biosensors based on living cells are highly sensitive to changes in the environment of the living cells, for example, changes in temperature and/or changes in the production batch of the bacteria. To overcome these drawbacks, a new measurable quantity was calculated, referred to herein as “standard ratio” (SR). The SR is defined as the ratio between the maximal output of the sensor exposed to an inspected sample contenting the target material and the maximal output of a control sample having a known amount, or a known concentration of the target material. In some embodiments, the new measurable quantity was found to be independent of batch and/or temperature influence.

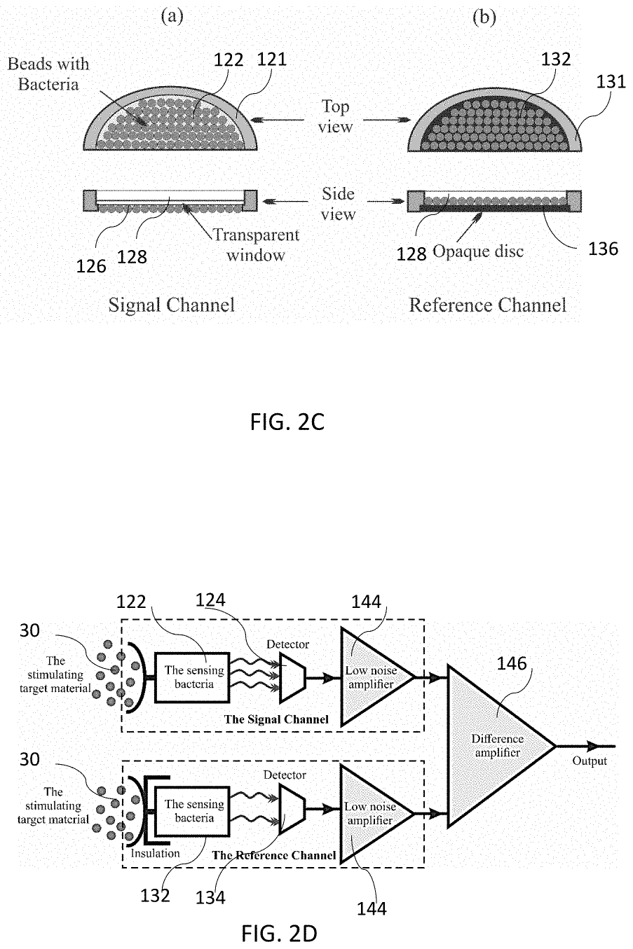

Reference is now made to FIG. 3A which is an illustration of a sensing unit for quantitatively detecting a target material according to some embodiments of the invention. A sensing unit 200 may include a housing 210 comprising a first compartment 220 and a second compartment 230. In some embodiments, first compartment 220 includes an inspection container 222 comprising genetically engineered living cells configured to produce a reporter when exposed to a target material, and a first detector 224 configured to detect said reporter. In some embodiments, second compartment 230 includes a control container 232 comprising the genetically engineered living cells configured to produce said reporter when exposed to the target material, and a second detector 134 configured to detect the reporter. In some embodiments, control container 232 also includes the known concentration or amount of the target material.

In some embodiments, inspection container 222 and control container 232 include the same amount or concentration of the genetically engineered living cells from the same production batch.

In a first non-limiting example, the reporter may be luminescent and the first detector 224 and second detector 234 are luminescence detectors configured to detect an emitted bioluminescent light, as discussed in the nonlimiting example, of FIG. 3B. In a second non-limiting example, the reporter may have chemiluminescence or bioluminescence. In a third non-limiting example, the reporter produces electricity or changes a conductivity of a material held in said inspection container and the first detector 224 and second detector 234 are any electrical property sensor including but not limited to: amperemeters, ohmmeters, LCR meters, voltmeters, or any device configured to measure, conductivity, resistivity, impedance, capacitance and the like. In a fourth non-limiting example, the reporter may be a chromogenic (color change) reporter, and first detector 224 and second detector 234 are selected from a camera and an optical detector.

In some embodiments, housing 210 further includes a blocking element 212 for blocking first compartment 220 from external biotic and abiotic cues, and/or blocking second compartment 230 from external biotic and abiotic cues.

In some embodiments, sensing unit 200 includes a control circuit 240 configured to receive signals from first detector 224 and second detector 234. In some embodiments, control circuit 240 may include additional components, such as signal amplifiers (e.g., low noise amplifiers, difference amplifiers, and the like), a processing unit (e.g., a chip), and the like. In some embodiments, sensing unit 200 includes a communication unit 250 configured to communicate with an external computing device (e.g., computing device 10 illustrated and discussed in FIGS. 4A and 5) and a power source 260 (e.g., a battery). In some embodiments, communication unit 250 may include any wireless or wired module.

In some embodiments, unit 200 may further include one or more heating and/or cooling elements. Therefore, such heating/cooling elements (not illustrated) may be integrated into housing 210.

Reference is now made to FIG. 3B which is an illustration of a non-limiting example of sensing unit 200 according to some embodiments of the invention. Sensing unit 200 of FIG. 2B includes two compartments 220 and 230 comprising inspection container 222 and control container 232 respectively. In the non-limiting example illustrated in FIG. 3B, the reporter is luminescent and first detector 224 and second detector 234 are luminescence detectors configured to detect emitted bioluminescent light.

In some embodiments, heating and/or cooling units may be integrated into housing 210 for controlling the temperature of the bacteria in the containers.

In the non-limiting example, the wavelength at which the bioluminescence emission is maximal is centered around 490 nm. Therefore, luminescence detectors 224 and 234 may be configured to detect photons having 490 nm wavelength. In some embodiments, the emitted light provides a weak signal (i.e., in the pico-watts range) therefore, requires amplification.

Sensing unit 200 may further include a digital unit 240, which may include an optoelectronic (OE) circuit 245 that detects and amplifies the optical signals emitted by the bacteria. OE circuit 245 produces an analog electronic signal that is proportional to the ratio (e.g., the SR) between the optical signals emitted by the bacteria (or the living cells) in first compartment 220 and second compartment 130.

In some embodiments, digital unit 240 is configured to process the output signal received from OE circuit 245 and optionally sends the signal to an external computing device via a communication unit 250. Communication unit 250 may include a wireless communication module and an antenna 255 for wirelessly communicating with the external computing device. In some embodiments, sensing unit 100 may further include one or more batteries 260 for providing electrical power to the various components of sensing unit 200 via powerline 265.

In some embodiments, the structure of sensing unit 200 allows each sensing unit to act as an independent, autonomous, standalone unit.

Reference is now made to FIG. 3C which is an illustration of a process for calculating the SR using sensing unit 200, according to some embodiments of the invention. Two solutions, having substantially the same volume, may be inserted into sensing unit 200. An inspected solution, that potentially includes an unknown amount and/or concentration of the target material, is inserted to inspection container 222 and a control solution is inserted to control container 232. The control solution comprises a known concentration or amount of the target material. Substantially the same amounts of genetically engineered living cells (e.g., bacteria) from the same batch may be inserted into the control container and the inspection container. The genetically engineered living cells are configured to generate a reporter when exposed to the target material.

Detector 224 may detect the generation of the reporter in inspection container 222, and detector 234 may detect the generation of the reporter in control container 232, for example, bioluminescence light emitted by the reporter may be detected by luminescence detectors. In some embodiments, the SR is the ratio between the maximal output signal generated by detector 224 and the maximal signal detected by detector 234.

### Sensing Unit System

In some embodiments, two or more sensing units 100 and/or 200 are included in a sensing system, as illustrated in FIG. 4A which is a block diagram of a system 300. System 300 further includes a computing device 10, for example, computing device 10 illustrated and discussed in FIG. 5. Each one of the sensing units 100 and/or 200 may communicate with computing device 10 wirelessly or by wired communication. For example, computing device 10 may receive from sensing unit 100 an output signal from difference amplifier 145 or receive from sensing unit 200 the SR. Computing device 10 may further process the received signals using any known methodology, for example, computing device 10 may compare the received signals, display the received signal (e.g., on a display), analyze the received signals, and the like.

Computing device 10 may further receive additional information related to each sensing unit 100 and/or 200, for example, the location (e.g., GPS coordinates) of each sensing unit. Computing device 10 may associate the signals received from each sensing unit with a location on a map and may display the received signal on the map. For example, when the target material is a chemical contaminant, potentially contaminating a specific area, computing device 10 may display on a map, location at which sensing units 100 detected a presence of this chemical.

Reference is now made to FIG. 4B which is a nonlimiting example for placement of sensing units in an area of interest according to some embodiments of the invention. In the non-limiting example of FIG. 4B, autonomous standalone sensing units 100 are placed at various places using drones.

Reference is now made to FIG. 5, which is a block diagram depicting a computing device, which may be included within an embodiment of a system of sensing units, according to some embodiments.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc.

Operating system 3 may be or may include any code segment (e.g., one similar to executable code 5 described herein) designed and/or configured to perform tasks involving coordination, scheduling, arbitration, supervising, controlling, or otherwise managing the operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read-only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under the control of operating system 3. For example, executable code 5 may be an application that may detect the presence and/or quantity of target material as further described herein. Although, for the sake of clarity, a single item of executable code 5 is shown in FIG. 5, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a microcontroller or chip as known in the art, a hard disk drive, a CD-Recordable (CD-R) drive, a Blu-ray disk (BD), a universal serial bus (USB) device or other suitable removable and/or fixed storage unit. In some embodiments, some of the components shown in FIG. 5 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse, and the like. Output devices 8 may include one or more (possibly detachable) displays or monitors, speakers, and/or any other suitable output devices. Any applicable input/output (I/O) devices may be connected to Computing device 10 as shown by blocks 7 and 8. For example, a wired or wireless network interface card (NIC), a universal serial bus (USB) device, or an external hard drive may be included in input devices 7 and/or output devices 8. It will be recognized that any suitable number of input devices 7 and output device 8 may be operatively connected to Computing device 10 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

### Methods of Detecting Target Materials

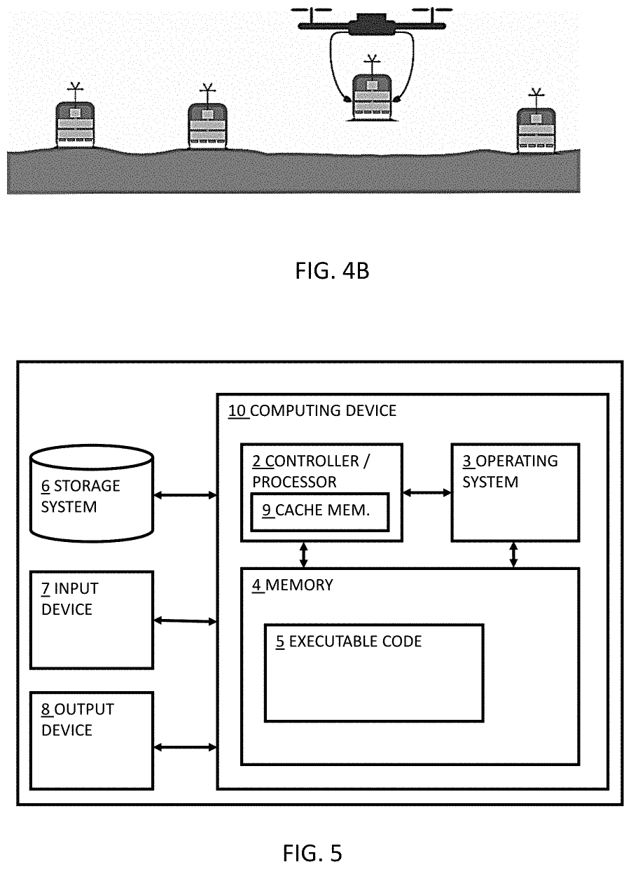

Reference is now made to FIG. 6, which is a flowchart of a method of sensing an environmental target material using genetically engineered living cells according to some embodiments of the invention. In some embodiment, environment or environmental is any medium, compound, composition, substance material, etc. derived from or present outside the: (a) sensing unit; or (b) the first compartment, the second compartment, or both, as described herein. The method may be performed by control circuit 140 and/or computing device 10 or by any other computing device. In step 610, a first signal indicative of a reporter may be received. The first signal may be received from a first detector 124 included in first compartment 120 comprising, the first group of polymer beads 122 as discussed hereinabove. In step 620, a second signal indicative of a spontaneous reaction or noise may be received from second detector 134 included in second compartment 130, comprising a second group of polymer beads 132, as discussed hereinabove. In some embodiments, the signal indicative of the reporter may be indicative of (i) generation of an electrical charge; (ii) emission of light produced by bioluminescence; (iii) chromogenic (color change) response; (iv) the production of fluorescent molecules (e.g. Green Fluorescence Protein (GFP)) and the like.

In step 630, the first signal is filtered using the second signal. For example, the second signal, indicating the spontaneous reaction or noise, is subtracted from the first signal, as illustrated in the non-limiting example of FIG. 2D. Alternatively, any filtering method may be used for filtering the first signal using the second signal. In some embodiments, filtering the first signal using the second signal may leave the first signal as indicative to reporter generated mainly due to the exposure to the target material.

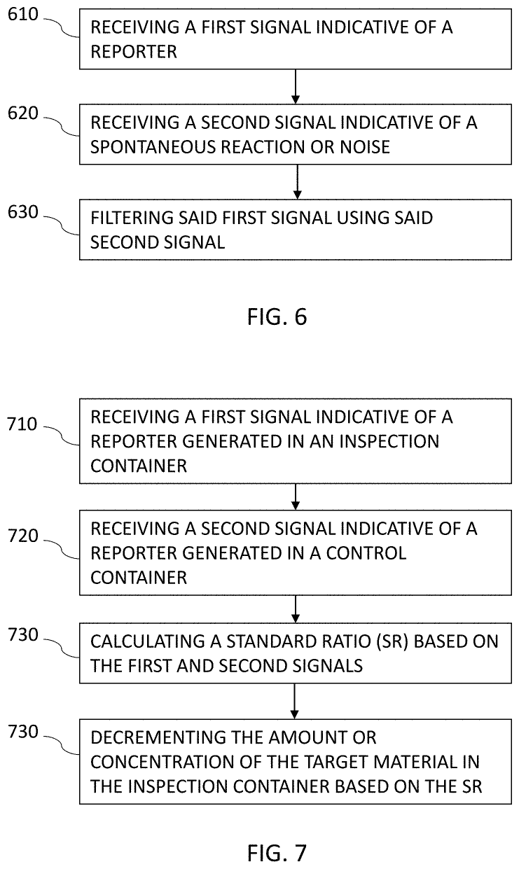

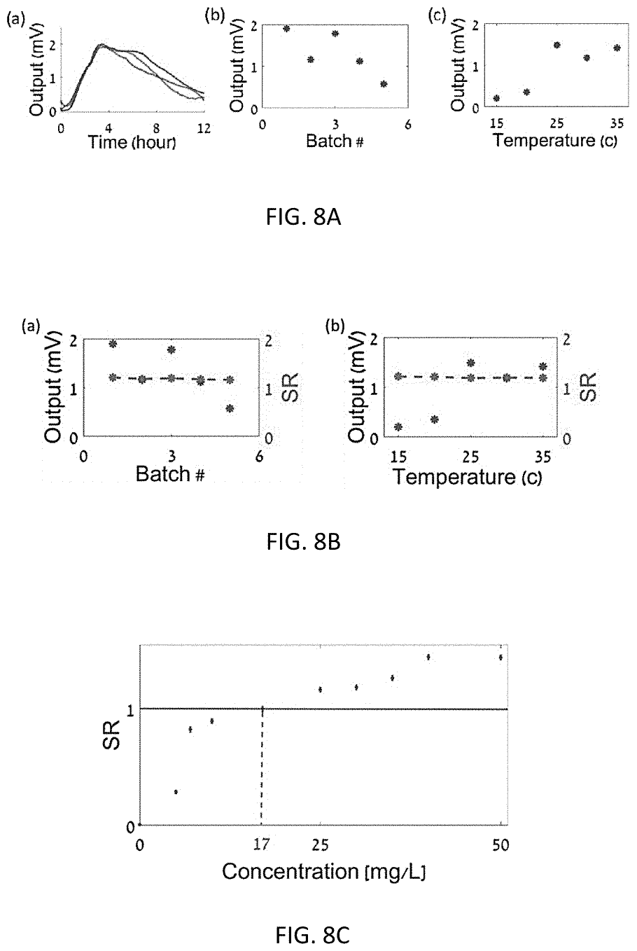

Reference is now made to FIG. 7 which is a method of quantitatively detecting an amount or concentration of a target material in a container, according to some embodiments of the invention. The method may be performed by control circuit 240 and/or computing device 10, or by any other computing device. In step 710, a first signal indicative of the generation of a reporter in inspection container 222 may be received from detector 224. In step 720, a second signal indicative of the generation of a reporter in control container 232 may be received from detector 234. In step 740, an SR may be calculated as the ratio between the maximum of the first signal (e.g., the maximal intensity) and the maximum of the second signal. Some nonlimiting examples for calculating SR are given in FIGS. 8A and 8B.

In step 740, the amount or concentration of the target material in the inspection container is determined based on the SR. For example, a calibration curve may be used to correlate the SR, the known amount, or concentration of the target material in the control container to the amount, or concentration of the target material on the inspection container. A non-limiting example, for calculating this calibration curve is given in FIG. 8C.

### EXAMPLES

### Quantitative Detection of Target Material

Quantitative detection of target material in a solution was performed using sensing unit 200 loaded with genetically engineered bacteria. The target material was 2,4-dinitrotoluene (DNT). Both inspection container 222 and control container 232 were loaded with 1 ml aqueous DNT solutions at different concentrations. An equal quantity of the bioluminescent bacteria and their nutrients were inserted into each of the inspected containers. The bioluminescent bacterium used was Escherichia coli strain P2G2, which responds to the presence of DNT. This strain hosts plasmid pACYC-yhaJ-G2, which harbors an enhanced version of the YhaJ transcriptional activator, obtained by several rounds of random mutagenesis. The highly conserved transcription factor acts as an activator of multiple genes among which is azoR, recently reported as a possible sensing element for the detection of DNT vapors. A mutated version of the azoR gene promoter, obtained by a single round of random mutagenesis was fused to a luxCDABE cassette of P. luminescens. The pazoR::luxCDABE fusion was mounted on a pBR2TTS backbone. An E. coli strain BW25113, deficient in the pykF gene, was used as a host due to its enhanced performance in the detection of DNT with yqjF promoter.

Results demonstrating the response, for a single compartment, of identical inspected solutions having the same amount of DNT and the same concentration of bacteria are presented in FIG. 8A. FIG. 8A (a) shows measurements of the signal output vs. time received from three identical solutions of 1 ml water with 30 mg/L DNT. In these measurements, the bacteria were from the same batch and the temperature at all measurements was identical. Thus, as can be seen in FIG. 8A (a), the maximum value of the output of all the measurements is similar.

FIG. 8A (b) shows the influence of using bacteria extracted from different batches. As clearly shown in FIG. 8A (b) there are significant variations in the output although the sampled solutions were chemically and biologically identical.

Major variations in the output obtained from containers operated at different ambient temperatures are shown in FIG. 8A (c). As demonstrated, the produced signal is batch- and/or temperature-dependent. Therefore, the raw bioluminescent signal, while dose-dependent, cannot be used as a direct measurement of the target material concentration and/or amount.

To overcome this drawback, the SR values were calculated using a control sample placed in control container 232 and inspection samples inserted to inspection container 222. The inspection samples were similar to the ones used for producing graphs 8A. The control sample contained 17 mg/L of DNT. The SR values and the output signal as a function of batch (graph (a)) and temperature (graph (b)) are presented in FIG. 8B. The SR results, marked as ‘*’ connected with a dashed line and the output results of FIG. 8A, are marked as discrete ‘*’. As can be seen, the SR is independent of both the bacterial batch and the operating temperature.

Accordingly, the SR can be used for determining the concentration and/or amount of target material in a solution, for example, a calibration curve of the SR vs. the control concentration can be used to assist with such a determination. An example of a calibration curve of DNT is presented in FIG. 8C. The calibration curve was extracted from a set of measurements performed with the sensing unit 200 presented in FIG. 3B. The DNT concentration of the standard sample was selected to be 17 mg\L due to the fact that this concentration lies in the middle of the dynamic range for which the bacterial sensor used in this work was found to be effective.

Using the calibration curve, the assessment of the concentration of the target material in the unknown inspected solution was performed by the following steps: measuring simultaneously for both samples the emitted bioluminescence over time; extracting the maximum value of the bioluminescent signal for each sample; calculating the SR by computing the ratio between the maximal value of the inspected sample and the maximal value of the control sample; comparing the SR values to a calibration curve, and calculating the concentration of the target material from the respective abscissa of the SR on the calibration curve.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A sensing unit comprising:
a housing, said housing comprises:
a first compartment comprising:
a first group of polymer beads comprising genetically engineered living cells configured to produce a reporter when exposed to a target material; and
a first detector configured to detect said reporter; and
a second compartment comprising:
a second group of said polymer beads comprising said genetically engineered living cells configured to produce said reporter when exposed to said target material; and
a second detector configured to detect said reporter;
wherein said second compartment is impenetrable to said target material and said first compartment is penetrable to said target material,
and wherein said first group of polymer beads and said second group of polymer beads comprise the same number of beads, and wherein all beads in both groups comprise an equal or similar distribution of said genetically engineered living cells from the same production batch.

2. The sensing unit of claim 1, wherein said second compartment comprises a cover, blocking said second group of said polymer beads from being exposed to said target material.

3. The sensing unit of claim 1, wherein said first compartment comprises an opening configured to allow said first group of polymer beads but not said second group of polymer beads to be exposed to said target material.

4. The sensing unit of claim 1, wherein said housing further comprises a blocking element for blocking at least said first compartment from external biotic and abiotic cues excluding said target material, said second compartment from external biotic and abiotic cues, or both.

5. The sensing unit of claim 1, further comprising:
a control circuit configured to receive a signal from said first detector and said second detector; and
a communication unit configured to communicate with an external computing device.

6. The sensing unit of claim 1, wherein said reporter luminescent and said first and said second detectors are luminescence detectors configured to detect an emitted bioluminescent light.

7. The sensing unit of claim 1, wherein said reporter changes an electrical property of a polymer bead and said first and said second detectors are selected from amperemeters, ohmmeters, LCR meters and voltmeters.

8. The sensing unit of claim 1, wherein said reporter chemiluminescence or bioluminescence.

9. The sensing unit of claim 1, designed to be placed on a ground surface.

10. A sensing unit comprising:
a housing said housing comprises;
a first compartment comprising:
an inspection container comprising genetically engineered living cells configured to produce a reporter when exposed to a target material; and
a first detector configured to detect said reporter; and
a second compartment comprising:
a control container comprising said genetically engineered living cells configured to produce said reporter when exposed to said target material; and
a second detector configured to detect said reporter;
wherein said control container comprising a known concentration or amount of said target material,
and wherein said inspection container and said control container comprise the same amount or concentration of genetically engineered living cells from the same production batch.

11. The sensing unit of claim 10, wherein said housing further comprises a blocking element for blocking said first compartment from external biotic and abiotic cues, said second compartment from external biotic and abiotic cues, or both.

12. The sensing unit of claim 10, further comprising:
a control circuit configured to receive signals from said first detector and said second detector; and
a communication unit configured to communicate with an external computing device.

13. The sensing unit of claim 10, wherein said reporter luminescent and said first and said second detectors are luminescence detectors configured to detect an emitted bioluminescent light.

14. The sensing unit of claim 10, wherein said reporter changes an electrical property of a material held in said inspection container and said control container and said first and said second detectors are selected from amperemeters, ohmmeters, LCR meters, and voltmeters.

15. The sensing unit of claim 10, wherein said reporter chemiluminescence or bioluminescence.

16. A method of sensing a target material using genetically engineered living cells, comprising:
receiving a first signal indicative of a reporter, wherein said first signal is from a first detector, said first detector is included in a first compartment comprising, a first group of polymer beads comprising genetically engineered living cells configured to produce a reporter when exposed to said target material;
receiving a second signal indicative of a spontaneous reaction or noise, wherein said second signal is from a second detector, said second detector is included in a second compartment, comprising, a second group of polymer beads comprising said genetically engineered living cell; and
filtering said first signal using said second signal,
wherein said second compartment is impenetrable to said target material and
said first compartment is penetrable to said target material,
and wherein said first group of polymer beads and said second group of polymer beads comprise the same number of beads, and wherein all beads in both groups comprise an equal or similar distribution of said genetically engineered living cells from the same production batch.

17. The method of claim 16, wherein said first detector and said second detector are configured to detect said reporter.
